(19) United States
(12) Patent Application Publication
(10) Pub. No.: US 2021/0299121 A1
(43) Pub. Date: Sep. 30, 2021

(54) CETIRIZINE OPHTHALMIC COMPOSITIONS

(71) Applicant: Somerset Therapeutics LLC, Hollywood, FL (US)

(72) Inventors: Mandar V. Shah, Somerset, NJ (US); Santhosh Kamath, Nelamangala (IN); Veerappan Subramanian, Somerset, NJ (US); Ilango Subramanian, Somerset, NJ (US)

(73) Assignee: Somerset Therapeutics LLC, Hollywood, FL (US)

(21) Appl. No.: 17/217,760

(22) Filed: Mar. 30, 2021

Related U.S. Application Data
(60) Provisional application No. 63/003,109, filed on Mar. 31, 2020.
(60) Provisional application No. 63/003,115, filed on Mar. 31, 2020.

(51) Int. Cl. A61K 31/495; A61K 47/10; A61K 47/38; A61K 47/26; A61K 47/18; A61K 47/02; A61K 9/08; A61K 9/00

(52) U.S. Cl. CPC A61K 31/495; A61K 47/10; A61K 47/38; A61K 9/0048; A61K 47/183; A61K 47/02; A61K 9/08; A61K 47/26

(57) ABSTRACT

The present invention relates to an ophthalmic composition comprising cetirizine and one or more pharmaceutically acceptable excipients, wherein said composition is free of benzalkonium chloride and/or dibasic sodium phosphate. The invention particularly relates to a 0.24% cetirizine ophthalmic composition, wherein said composition is free of benzalkonium chloride and/or dibasic sodium phosphate. Further, the invention may also provide a process of preparing such composition and their use for treating allergic conjunctivitis and/or allergic rhinoconjunctivitis.

### FIELD OF THE INVENTION

Disclosed herein are ophthalmic compositions comprising cetirizine, particularly compositions which are free of benzalkonium chloride and/or dibasic sodium phosphate. The present invention also relates to a process of preparing such compositions and their use for treating allergic conjunctivitis and/or allergic rhinoconjunctivitis.

### BACKGROUND OF THE INVENTION

Cetirizine hydrochloride is a racemic selective H1 receptor agonist which functions as an antihistamine. Cetirizine hydrochloride is (RS)-2-[2-[4-[(4-Chlorophenyl) phenylmethyl] piperazin-1-yl] ethoxy] acetic acid, dihydrochloride. The chemical structure is as follows:

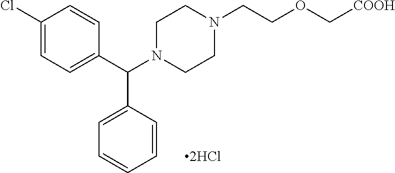 [SMILES: O=C(O)COCCN1CCN(C(c2ccccc2)c2ccc(Cl)cc2)CC1]

Allergic conjunctivitis is an inflammation of the conjunctiva due to allergy, primarily due to hay fever. The symptoms consist of redness, edema of the conjunctiva, itching, and increased lacrimation. If this is combined with rhinitis, the condition is referred to as allergic rhinoconjunctivitis. These symptoms are due to release of histamine and other active substances by mast cells, which stimulate dilation of blood vessels, irritate nerve endings, and increase secretion of tears. Allergic conjunctivitis and rhinoconjunctivitis may also co-exist with other external ocular conditions and diseases, such as dry eye, or irritations caused by pollutants or other causes.

Cetirizine hydrochloride is FDA approved for oral use and is used as a systemic antihistamine for the treatment of allergies, hay fever, angioedema, and urticaria. Oral antihistamines have been shown to induce decreased tear production and lead to dryness of the ocular surface, which can exacerbate ocular discomfort and can make the eye susceptible to irritation by an ophthalmic product.

Currently available treatments for eye allergy include artificial tears drops, which can wash allergens off the ocular surface and act as a barrier for the eye; antihistamines, which block histamine from binding to the histamine receptors; mast cell stabilizers that block the release of histamine and other substances from the mast cell; vasoconstrictors that can actively constrict blood vessels thus reducing redness and swelling; and drugs with multiple modes of action, for example antihistamine/mast cell stabilizing agents. The criteria which may be considered in evaluating the appropriateness of an agent for a patient include: efficacy at onset of action, duration of action, how well it controls the individual signs and symptoms of allergic conjunctivitis, comfort of the formulation when instilled in the eye, and safety of the formulation when instilled in the eye. The various available treatments include ophthalmic drops/solution, nasal sprays, and systemic oral agents.

It has been difficult to prepare cetirizine as an ophthalmic solution with satisfactory safety and stability profiles as it has a poor stability at concentrations less than 1% (w/v) and at higher concentrations (1% and above) is strongly irritating and thus unsuitable for ocular administration.

U.S. Pat. No. 5,419,898 discloses use of a cyclodextrin compound to increase the solubility and stability of cetirizine for ophthalmic use.

A cetirizine hydrochloride ophthalmic solution at a concentration of 0.24% is currently approved for marketing under the brand name Zerviate® by Eyevance Pharmaceuticals. Zerviate® is indicated for the treatment of ocular itching associated with allergic conjunctivitis. The approved composition contains cetirizine 2.40 mg (equivalent to 2.85 mg of cetirizine hydrochloride) as an active ingredient and the following inactive ingredients: benzalkonium chloride 0.01% (preservative); glycerin; sodium phosphate, dibasic; edetate disodium; polyethylene glycol 400; polysorbate 80; hypromellose; hydrochloric acid/sodium hydroxide (to adjust pH); and water for injection. The composition and its use are covered by U.S. Pat. Nos. 8,829,005; 9,254,286; 9,750,684 and 9,993,471.

However, use of benzalkonium chloride at higher concentrations can be undesirable to the ocular cells. Eye drops preserved with benzalkonium chloride, as compared to preservative-free eye drops, may induce ocular symptoms and signs of irritation in patients, such as pain or discomfort, foreign body sensation, stinging or burning, and dry eye sensation. As such, the inventors have determined that there is a benefit to be attained by avoiding the use of benzalkonium chloride in a cetirizine ophthalmic formulation. However, benzalkonium chloride is a well-known and characterized preservative that is readily accepted in the pharmaceutical arts. It is expected that avoiding its use in an ophthalmic formulation will be difficult.

Patients experiencing hypersensitivity reactions with benzalkonium chloride cannot use a commercial cetirizine product preserved with 0.01% benzalkonium chloride. B enzalkonium chloride also may be absorbed by the soft contact lenses therefore patients wearing soft contact lenses are advised to remove lenses prior to administration and wait at least 15 minutes before reinserting them.

Thus, the inventors have determined that there is an enduring need to develop an improved cetirizine ophthalmic composition that provides an alternative to existing formulations for treating allergic conjunctivitis and/or allergic rhinoconjunctivitis. The inventors of the present invention have developed cetirizine ophthalmic compositions, wherein the compositions are free of benzalkonium chloride and/or dibasic sodium phosphate. Also, the inventors have developed cetirizine hydrochloride ophthalmic compositions using benzalkonium chloride at a level less than 0.05 ppm and/or another nontoxic preservative, which is free of dibasic sodium phosphate. The composition prepared by using the current invention exhibits good physical and chemical stability.

### SUMMARY OF THE INVENTION

The present invention provides an ophthalmic composition comprising cetirizine for treating allergic conjunctivitis and/or allergic rhinoconjunctivitis. In particular, the present invention provides a cetirizine ophthalmic solution, wherein the solution is free of benzalkonium chloride and/or dibasic sodium phosphate.

In one general aspect, there is provided an ophthalmic composition comprising cetirizine, wherein said composition is free of benzalkonium chloride and/or dibasic sodium phosphate.

In one general aspect, there is provided an ophthalmic composition comprising cetirizine at a concentration of about 0.01% to about 1.0% w/v, wherein said composition is free of benzalkonium chloride and/or dibasic sodium phosphate.

In one general aspect, there is provided an ophthalmic composition comprising cetirizine at a concentration of about 0.24% w/v, wherein said composition is free of benzalkonium chloride and/or dibasic sodium phosphate.

In another general aspect, there is provided an ophthalmic composition comprising about 0.24% cetirizine and one or more pharmaceutically acceptable excipients, wherein said composition is free of benzalkonium chloride and/or dibasic sodium phosphate.

In another general aspect, there is provided an ophthalmic composition comprising about 0.24% cetirizine, viscosity-enhancer, chelating agent, tonicity agents, buffers or pH-adjusting agent, preservatives and water.

In another general aspect, there is provided an ophthalmic composition comprising about 0.24% cetirizine, viscosity-enhancer, chelating agent, tonicity agents, buffers or pH-adjusting agent, preservatives and water, wherein said composition is free of benzalkonium chloride and/or dibasic sodium phosphate.

In another general aspect, there is provided an ophthalmic composition comprising about 0.24% cetirizine and one or more pharmaceutically acceptable excipients selected from viscosity-enhancer, chelating agent, tonicity agents, buffers or pH-adjusting agent, preservatives and water, wherein said composition is free of benzalkonium chloride and/or dibasic sodium phosphate. The amount and type of excipient added is in accordance with the particular requirements of the composition and is generally in the range of from about 0.0001% to 90% by weight.

In another general aspect, there is provided an ophthalmic composition comprising about 0.24% cetirizine and one or more pharmaceutically acceptable excipients, wherein said composition comprises less than 50 ppm of benzalkonium chloride.

In another general aspect, there is provided an ophthalmic composition comprising about 0.24% cetirizine and one or more pharmaceutically acceptable excipients, wherein said composition comprises less than 50 ppm of benzalkonium chloride and/or is free of dibasic sodium phosphate.

In another general aspect, there is provided an ophthalmic composition comprising about 0.24% cetirizine and one or more pharmaceutically acceptable excipients, wherein said composition comprises nontoxic preservatives other than benzalkonium chloride.

In another general aspect, there is provided an ophthalmic composition comprising about 0.24% cetirizine and one or more pharmaceutically acceptable excipients, wherein said composition comprises nontoxic preservatives other than benzalkonium chloride and/or is free of dibasic sodium phosphate.

In another general aspect, there is provided an ophthalmic composition comprising about 0.24% cetirizine and one or more pharmaceutically acceptable excipients, wherein said composition comprises nontoxic preservatives selected from zinc chloride, sodium chlorite, sodium hydroxymethyl glycinate, polyquaternium-1 and phenylethyl alcohol, alone or in combination thereof.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium and water, wherein said composition further comprises nontoxic preservatives selected from zinc chloride, sodium chlorite, sodium hydroxymethyl glycinate, polyquaternium-1 and phenylethyl alcohol, alone or in combination thereof and buffers selected from boric acid, sodium borate, citric acid alone or in combination thereof and said composition is free of benzalkonium chloride.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium and water, wherein said composition further comprises nontoxic preservatives selected from zinc chloride, sodium chlorite, sodium hydroxymethyl glycinate, polyquaternium-1 and phenylethyl alcohol, alone or in combination thereof and one or more buffers selected from boric acid, sodium borate, and citric acid alone or in combination thereof and said composition is free of benzalkonium chloride and dibasic sodium phosphate.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.005% zinc chloride, about 0.2% boric acid, 0.2% sodium borate and water, wherein said composition is free of benzalkonium chloride and dibasic sodium phosphate.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.001% sodium chlorite, about 0.2% boric acid, 0.2% sodium borate and water, wherein said composition is free of benzalkonium chloride and dibasic sodium phosphate.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.001% polyquaternium-1, about 0.2% boric acid, 0.2% sodium borate and water, wherein said composition is free of benzalkonium chloride and dibasic sodium phosphate.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.25% phenylethyl alcohol, about 0.2% boric acid, 0.2% sodium borate and water, wherein said composition is free of benzalkonium chloride and dibasic sodium phosphate.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.002% sodium hydroxymethyl glycinate, about 0.2% boric acid, 0.2% sodium borate and water, wherein said composition is free of benzalkonium chloride and dibasic sodium phosphate.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.005% zinc chloride, about 0.2% boric acid, 0.2% sodium borate and water, wherein said composition is free of benzalkonium chloride.

In another general aspect, there is provided an ophthalmic composition comprising about 0.24% cetirizine and one or more pharmaceutically acceptable excipients selected from viscosity-enhancer, chelating agent, tonicity agents, buffers or pH-adjusting agent and water, wherein the composition is filled in preservative free bottle.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium and water, wherein said composition further comprises buffers selected from boric acid, sodium borate, citric acid alone or in combination thereof, wherein the composition is free of preservative.

In another general aspect, there is provided an ophthalmic composition comprising about 0.24% cetirizine, about 1% polyethylene glycol, about 0.25% hypromellose, about 0.1% polysorbate 80, about 1.8% glycerin, about 0.025% edetate sodium and water, wherein said composition further comprises buffers selected from boric acid, sodium borate, citric acid alone or in combination thereof and the composition is filled in preservative free bottle.

In another general aspect, there is provided an ophthalmic composition comprising about 0.24% cetirizine, about 1% polyethylene glycol, about 0.25% hypromellose, about 0.1% polysorbate 80, about 0.2% boric acid, about 0.2% sodium borate, about 1.8% glycerin, about 0.025% edetate sodium and water, wherein said composition is filled in preservative free bottle.

In another general aspect, there is provided an ophthalmic composition comprising about 0.24% cetirizine, about 1% polyethylene glycol, about 0.25% hypromellose, about 0.1% polysorbate 80, about 0.2% boric acid, about 0.2% sodium borate, 0.005% or less benzalkonium chloride, about 1.8% glycerin, about 0.025% edetate sodium and water, wherein said composition is filled in preservative free bottle.

In another general aspect, there is provided an ophthalmic composition comprising about 0.24% cetirizine by weight and one or more pharmaceutically acceptable excipients, wherein said composition is free of benzalkonium chloride and/or dibasic sodium phosphate and applied once or twice daily in the affected eye(s).

In another general aspect, there is provided a process for the preparation of an ophthalmic composition comprising about 0.24% cetirizine by weight, wherein said composition is free of benzalkonium chloride and/or dibasic sodium phosphate.

In another general aspect, there is provided a process for preparing and sterilizing the compositions in order to reduce the amounts of related compounds and impurities associated with the ophthalmic compositions on storage.

In another general aspect, there is provided a process for preparation of an ophthalmic composition comprising about 0.24% cetirizine and one or more pharmaceutically acceptable excipients, wherein said composition comprises nontoxic preservatives selected from zinc chloride, sodium chlorite, sodium hydroxymethyl glycinate, polyquaternium-1 and phenylethyl alcohol, alone or in combination thereof.

In another general aspect, there is provided a process for preparation of an ophthalmic composition comprising about 0.24% cetirizine, about 1% polyethylene glycol, about 0.25% hypromellose, about 0.1% polysorbate 80, about 1.8% glycerin, about 0.025% edetate sodium and water, wherein said composition further comprises nontoxic preservatives selected from zinc chloride, sodium chlorite, sodium hydroxymethyl glycinate, polyquaternium-1 and phenylethyl alcohol, alone or in combination thereof and buffers selected from boric acid, sodium borate, and citric acid alone or in combination thereof and said composition is free of benzalkonium chloride.

In another general aspect, there is provided a process for preparation of an ophthalmic composition comprising about 0.24% cetirizine, about 1% polyethylene glycol, about 0.25% hypromellose, about 0.1% polysorbate 80, about 1.8% glycerin, about 0.025% edetate sodium and water, wherein said composition further comprises nontoxic preservatives selected from zinc chloride, sodium chlorite, sodium hydroxymethyl glycinate, polyquaternium-1 and phenylethyl alcohol, alone or in combination thereof and buffers selected from boric acid, sodium borate, citric acid alone or in combination thereof and said composition is free of benzalkonium chloride and dibasic sodium phosphate.

In another general aspect, there is provided a process for preparation of an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.005% zinc chloride, about 0.2% boric acid, 0.2% sodium borate and water, wherein said composition is free of benzalkonium chloride and dibasic sodium phosphate.

In another general aspect, there is provided a process for preparation of an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.001% sodium chlorite, about 0.2% boric acid, 0.2% sodium borate and water, wherein said composition is free of benzalkonium chloride and dibasic sodium phosphate.

In another general aspect, there is provided a process for preparation of an ophthalmic composition comprising about 0.24% cetirizine, about 1% polyethylene glycol, about 0.25% hypromellose, about 0.1% polysorbate 80, about 1.8% glycerin, about 0.025% edetate sodium, about 0.001% polyquaternium-1, about 0.2% boric acid, 0.2% sodium borate and water, wherein said composition is free of benzalkonium chloride and dibasic sodium phosphate.

In another general aspect, there is provided a process for preparation of an ophthalmic composition comprising about 0.24% cetirizine, about 1% polyethylene glycol, about 0.25% hypromellose, about 0.1% polysorbate 80, about 0.2% boric acid, about 0.2% sodium borate, 0.005% or less benzalkonium chloride, about 1.8% glycerin, about 0.025% edetate sodium and water, wherein said composition is filled in preservative free bottle.

By using a preservative-free bottle, the sterility of the composition contained within can be enhanced. In this manner, less preservative need be included in the composition. For example, by using a preservative-free bottle, less benzalkonium chloride is necessary. In some compositions in a preservative-free bottle, no benzalkonium chloride is included and the composition remains suitably sterile for its shelf life. By appropriate sterilization of the composition before being filled in the preservative-free bottle, the sterility of the composition contained within can have its sterility improved.

The sterilization is carried out by using one or more methods selected from heat sterilization, gaseous sterilization, filtration sterilization or radiation sterilization.

In another general aspect, there is provided an ophthalmic formulation of cetirizine in combination with an additional active agent such as a steroid or a vasoconstrictor.

In another general aspect, there is provided an ophthalmic composition comprising about 0.24% cetirizine by weight, wherein the composition is free of benzalkonium chloride and/or dibasic sodium phosphate and characterized in that the dosage form retains at least 90% w/w of the potency of cetirizine when stored at 25° C. and 60% relative humidity or at 40° C. and 75% relative humidity for 3 months.

### DETAILED DESCRIPTION OF THE INVENTION

The invention provides an ophthalmic composition comprising cetirizine hydrochloride. In particular, the invention relates to an ophthalmic composition comprising cetirizine hydrochloride, wherein the composition is free of benzalkonium chloride and/or dibasic sodium phosphate. Further, the invention also relates to an ophthalmic composition comprising cetirizine hydrochloride and benzalkonium chloride at a level less than 0.05 ppm and/or other nontoxic preservative, which is free of dibasic sodium phosphate.

The term “cetirizine” used throughout the specification refers to not only cetirizine per se, but also its other pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

The ophthalmic composition of cetirizine comprises cetirizine at a concentration range of from about 0.01% to 1.0% by weight, preferably 0.05% to 0.5%, or any specific value within said ranges. Preferably, cetirizine is in the form of cetirizine hydrochloride or dihydrochloride.

The ophthalmic composition according to the present invention may be in the form of a solution, emulsion, dispersion, suspension, reverse emulsion and microemulsion.

The one or more pharmaceutically acceptable excipients used in the ophthalmic compositions include but are not limited to one or more of a thickening agent or viscosity-enhancer, solubilizer, penetration enhancer, chelating agent, tonicity agent, buffer or pH-adjusting agent, preservative and water.

The solubilizer and the penetration enhancer can be the same or different. Exemplary solubilizers and penetration enhancers include, but are not limited to, polysorbate 80, tocopheryl polyethylene glycol succinate (TPGS), polyoxyl 35 castor oil, polyarginine, polycerine, tromethamine (tris), and sesame seed oil. The solubilizer and the penetration enhancer can be present in an amount of about 0.5% to 0.2% by weight.

Viscosity-enhancing agents are used in the ophthalmic compositions to improve the form of the formulation for convenient administration and to improve contact with the eye and thereby improve bioavailability. Exemplary thickening agents include, but are not limited to, polymers containing hydrophilic groups such as monosaccharides and polysaccharides, ethylene oxide groups, hydroxyl groups, carboxylic acids or other charged functional groups. While not intending to limit the scope of the invention, some examples of useful viscosity-enhancing agents are hydroxypropyl methylcellulose (HPMC or hypromellose), sodium carboxymethylcellulose, povidone, polyvinyl alcohol, and polyethylene glycol. The viscosity-enhancing agents can be present from about 0.1% to about 2% by weight, or any specific value within said range, preferably from about 0.1% to about 0.5%.

Tonicity agents are used in the ophthalmic compositions to adjust the composition of the formulation to be within a desired isotonic range. Exemplary tonicity agents include, but are not limited to, glycerin, mannitol, sorbitol, sodium chloride, and other electrolytes. The tonicity agents are in the amount of about 0.01% to 0.8% by weight. The osmolality of the composition is of about 250 to about 350 mOsm/kg, preferably about 300 mOsm/kg.

Chelating agents are used in the ophthalmic compositions to enhance preservative effectiveness by forming stable water-soluble complexes (chelates) with alkaline earth and heavy metal ions. Exemplary chelating agents include, but are not limited to, ethylenediaminetetraacetic acid (EDTA), or salts thereof. The chelating agent typically is present in an amount from about 0.001-0.1% by weight. In the case of EDTA, the chelating agent is preferably present at a concentration of about 0.025% by weight.

Buffers or pH-adjusting agents in the ophthalmic compositions are used to adjust the pH to a desirable range. Exemplary buffers include, but are not limited to, boric acid, sodium borate, potassium citrate, citric acid, sodium bicarbonate and TRIS buffers alone or in combination thereof. The pH of the present solutions should be maintained within the range of 4.0 to 8.0, more preferably about 5.5 to 7.5, more preferably about 7.0. The amount of buffers used in the composition ranges from about 0.05% to about 2.5% by weight, and preferably from about 0.1% to about 1.0%.

Preservatives in the ophthalmic compositions are used to inhibit microbial growth. Suitable nontoxic preservatives include, but are not limited to, zinc chloride, sodium chlorite, sodium hydroxymethyl glycinate, polyquaternium compound such as polyquaternium-1, cationic compounds such as chlorhexidine gluconate, p-hydroxybenzoates such as methyl p-hydroxybenzoate, ethyl p-hydroxybenzoate, propyl p-hydroxybenzoate and butyl phydroxybenzoate, alcohol compounds such as phenylethyl alcohol, benzyl alcohol and chlorobutanol, sodium dehydroacetate; amino acids such as cysteine and methionine, citric acid and sodium citrate and other preservatives such as thimerosal, octadecyldimethylbenzyl ammonium chloride, hexamethonium chloride, benzethonium chloride, phenol, catechol, resorcinol, cyclohexanol, 3-pentanol, m-cresol, phenylmercuric nitrate, phenylmercuric acetate or phenylmercuric borate, sodium perborate and the like either alone or in combination thereof. The amount of preservative used in the composition ranges from about 0.01% to about 0.1% by weight.

The cetirizine ophthalmic composition without benzalkonium chloride will improve efficacy of the composition with superior patient compliance and fewer side effects than the cetirizine composition with benzalkonium chloride. The side effects which may be avoided with the preservative free composition include visual disturbance, ocular burning, foreign body sensation, eye pain, eyelid erythema, ocular irritation, eye discharge, tearing, photophobia, allergic conjunctivitis, asthenopia, conjunctival edema, conjunctival hemorrhage, and intraocular inflammation.

The ophthalmic compositions of cetirizine (e.g., solutions) without benzalkonium chloride are believed to offer advantages, such as a superiority to current compositions from a safety, tolerability and patient compliance standpoint while maintaining and/or improving its efficacy. Further, the compositions would be the first available for use by those patients who are hypersensitive to benzalkonium chloride and as well as offering convenience for patients wearing soft contact lenses.

The ophthalmic composition of cetirizine can be provided in combination with an additional active agent such as a vasoconstrictor or a steroid. The combination formulations of cetirizine are effective in mitigating the signs and symptoms of both acute and late phase allergic conjunctivitis, such as ocular itching, redness, chemosis, and lid swelling, and nasal symptoms, as well as allergic rhinoconjunctivitis.

It is to be understood that the above description is intended to be illustrative and not restrictive. Many embodiments will be apparent to those of skill in the art upon reading the above description. The scope of the invention should, therefore, be determined with reference to the appended claims, along with the full scope of equivalents to which such claims are entitled. The disclosures of all articles and references, including patents, patent applications and publications, are incorporated herein by reference in their entirety and for all purposes.

### A. Preservative Free Formulation

### Example 1

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Polysorbate 80 | 0.7 to 1.3 mg |
| 7 | Glycerin | 1.0 to 4.0 mg |
| 8 | Edetate sodium | 0.1 to 0.5 mg |
| 9 | Sodium hydroxide NF (0.1N) | q.s. to adjust pH |
| | | 7.0 ± 0.2 |
| 10 | Hydrochloric acid NF (0.1N) | q.s. to adjust pH |
| | | 7.0 ± 0.2 |
| 11 | Water | q.s. to 1 mL |

### Example 2

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Sodium Citrate | 1.0 to 4.0 mg |
| 5 | Polysorbate 80 | 0.7 to 1.3 mg |
| 6 | Glycerin | 1.0 to 4.0 mg |
| 7 | Edetate sodium | 0.1 to 0.5 mg |
| 8 | Sodium hydroxide NF (0.1N) | q.s. to adjust pH |
| | | 7.0 ± 0.2 |
| 9 | Hydrochloric acid NF (0.1N) | q.s. to adjust pH |
| | | 7.0 ± 0.2 |
| 10 | Water | q.s. to 1 mL |

Manufacturing Process: The manufacturing process has the following steps:

Part I
1. In a suitable container, take 30% of required water for injection (WFI) and bring it to 70° C., under nitrogen purging.
2. Add the required quantity of hypromellose with stirring to form a hypromellose dispersion.
3. Sterilize the hypromellose dispersion at about 121° C. for about 30 minutes and cool it to hydrate the hypromellose.

Part II
4. In another container, take 50% of the required WFI and cool it to room temperature under nitrogen purging.
5. To this container of cooled WFI, add the remaining ingredients, except for the cetirizine HCl and pH adjusting agents, one after the other, ensuring that the previous ingredient is dissolved first before adding the next ingredient.
6. Mix the obtained solution and adjust its pH to 7.0±0.2 by using 0.1 N HCl or 0.1 N NaOH.
7. Add the drug, Cetirizine HCl, to the solution of step 6 with stirring to dissolve it completely.

Part III
8. Sterile filter 30% of the required water and cool it under nitrogen purging.

Part IV
9. Sterile filter the solution of Part II into a sterile tank (tank IV) and rinse the line with 10% of the extra water from Part III.
10. Pass the solution of Part I through a sterile 8-micron polyether sulfone filter into tank IV.
11. Rinse and adjust the final volume in tank IV with the remaining sterile water from Part III.
12. Stir the solution and fill the product into appropriate sterile ophthalmic containers. Please note, excess quantity of Part III may be prepared as needed.

### B. Formulation with Non-Toxic Preservative (BKC Free)

### Example 1

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Sodium Chlorite (NaClO₂) | 0.005 to 0.02 mg |
| 7 | Polysorbate 80 | 0.7 to 1.3 mg |
| 8 | Glycerin | 1.0 to 4.0 mg |
| 9 | Edetate sodium | 0.1 to 0.5 mg |
| 10 | Sodium hydroxide NF (0.1N) | q.s. to adjust pH |
| | | 7.0 ± 0.2 |
| 11 | Hydrochloric acid NF (0.1N) | q.s. to adjust pH |
| | | 7.0 ± 0.2 |
| 12 | Water | q.s. to 1 mL |

### Example 2

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Sodium Chlorite (NaClO₂) | 0.005 to 0.02 mg |
| 7 | Zinc Chloride | 0.025 to 0.1 mg |
| 8 | Polysorbate 80 | 0.7 to 1.3 mg |
| 9 | Glycerin | 1.0 to 4.0 mg |
| 10 | Edetate sodium | 0.1 to 0.5 mg |
| 11 | Sodium hydroxide NF (0.1N) | q.s. to adjust pH |
| | | 6.0 ± 0.2* |
| 12 | Hydrochloric acid NF (0.1N) | q.s. to adjust pH |
| | | 6.0 ± 0.2* |
| 13 | Water | q.s. to 1 mL |

*Whenever, zinc chloride is present, the pH should be around 6.0. Otherwise, the solution will turn hazy.

### Example 3

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Sodium Chlorite (NaClO₂) | 0.005 to 0.02 mg |
| 7 | Zinc Chloride | 0.025 to 0.1 mg |
| 8 | Polysorbate 80 | 0.7 to 1.3 mg |
| 9 | Glycerin | 1.0 to 4.0 mg |
| 10 | Edetate sodium | 0.1 to 0.5 mg |
| 11 | Sodium hydroxide NF (0.1N) | q.s. to adjust pH |
| | | 6.0 ± 0.2 |
| 12 | Hydrochloric acid NF (0.1N) | q.s. to adjust pH |
| | | 6.0 ± 0.2 |
| 13 | Water | q.s. to 1 mL |

### Example 4

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | polyquaternium-1 | 0.005 to 0.1 mg |
| 7 | Polysorbate 80 | 0.7 to 1.3 mg |
| 8 | Glycerin | 1.0 to 4.0 mg |
| 9 | Edetate sodium | 0.1 to 0.5 mg |
| 10 | Sodium hydroxide NF (0.1N) | q.s. to adjust pH |
| | | 7.0 ± 0.2 |
| 11 | Hydrochloric acid NF (0.1N) | q.s. to adjust pH |
| | | 7.0 ± 0.2 |
| 12 | Water | q.s. to 1 mL |

### Example 5

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Benzalkonium chloride | 0.005 mg |
| 7 | Polysorbate 80 | 0.7 to 1.3 mg |
| 8 | Glycerin | 1.0 to 4.0 mg |
| 9 | Edetate sodium | 0.1 to 0.5 mg |
| 10 | Sodium hydroxide NF (0.1N) | q.s. to adjust pH |
| | | 7.0 ± 0.2 |
| 11 | Hydrochloric acid NF (0.1N) | q.s. to adjust pH |
| | | 7.0 ± 0.2 |
| 12 | Water | q.s. to 1 mL |

### Example 6

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Phenylethyl alcohol USP | 1.0 to 5.0 mg |
| 7 | Polysorbate 80 | 0.7 to 1.3 mg |
| 8 | Glycerin | 1.0 to 4.0 mg |
| 9 | Edetate sodium | 0.1 to 0.5 mg |
| 10 | Sodium hydroxide NF (0.1N) | q.s. to adjust pH |
| | | 7.0 ± 0.2 |
| 11 | Hydrochloric acid NF (0.1N) | q.s. to adjust pH |
| | | 7.0 ± 0.2 |
| 12 | Water for Injection | q.s. to 1 mL |

### Example 7

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Sodium Hydroxymethyl Glycinate | 0.01 to 1.0 mg |
| 7 | Polysorbate 80 | 0.7 to 1.3 mg |
| 8 | Glycerin | 1.0 to 4.0 mg |
| 9 | Edetate sodium | 0.1 to 0.5 mg |
| 10 | Sodium hydroxide NF (0.1N) | q.s. to adjust pH |
| | | 7.0 ± 0.2 |
| 11 | Hydrochloric acid NF (0.1N) | q.s. to adjust pH |
| | | 7.0 ± 0.2 |
| 12 | Water for Injection | q.s. to 1 mL |

### Example 8

| Sr. | | Quantity/mL |
|---|---|---|
| No | Name of ingredients | Ranges |
| 1 | Cetirizine HCl equivalent to 0.24% | None |
| 2 | Polyethylene glycol, 400 | 5 to 20 mg |
| 3 | Hypromellose, USP 2910 | 1.5 to 5 mg |
| 4 | Boric Acid, USP | 1.0 to 4.0 mg |
| 5 | Sodium Borate, USP | 1.0 to 4.0 mg |
| 6 | Zinc Chloride | 0.025 to 0.1 mg |
| 7 | Polysorbate 80 | 0.7 to 1.3 mg |
| 8 | Glycerin | 1.0 to 4.0 mg |
| 9 | Edetate sodium | 0.1 to 0.5 mg |
| 10 | Sodium hydroxide NF (0.1N) | q.s. to adjust pH |
| | | 7.0 ± 0.2 |
| 11 | Hydrochloric acid NF (0.1N) | q.s. to adjust pH |
| | | 7.0 ± 0.2 |
| 12 | Water for Injection | q.s. to 1 mL |

Manufacturing Process: The manufacturing process has the following steps:

Part I
1. In a suitable container, take 30% of required water for injection (WFI) and bring it to 70° C., under nitrogen purging.
2. Add the required quantity of hypromellose with stirring to form a hypromellose dispersion.
3. Sterilize the resulting hypromellose dispersion at about 121° C. for about 30 minutes and cool it to hydrate the hypromellose.

Part II
4. In another container, take 50% of the required WFI and cool it to room temperature under nitrogen purging.
5. To this container of cooled WFI, add the remaining ingredients, except the cetirizine HCl and pH adjusting agents, one after the other, ensuring that the previous ingredient is dissolved first before adding the next ingredient.
6. Mix the obtained solution and adjust its pH to 7.0±0.2 by using 0.1 N HCl or 0.1 N NaOH.
7. Add the drug, Cetirizine HCl, to the solution of step 6 with stirring to dissolve it completely.

Part III
8. Sterile filter 30% of the required water and cool it under nitrogen purging.

Part IV
9. Sterile filter the solution of Part II into a sterile tank (tank IV) and rinse the line with 10% of the extra water from Part III.
10. Pass the solution of Part I through sterile 8-micron polyether sulfone filter into tank IV.
11. Rinse and adjust the final volume of tank IV with the remaining water from Part III.
12. Stir the solution and till the product into appropriate sterile ophthalmic containers.
13. Please note, excess quantity of Part III may be prepared as needed.

The compositions described herein are tested for stability including one or more of appearance (contents and container integrity), assay for label claim of cetirizine, pH, osmolality, sterility, particulate matter, and antimicrobial preservative efficacy. The stability conditions can include 25° C./60% relative humidity, 40° C./75% relative humidity, 25° C./40% relative humidity, and/or 40° C./25% relative humidity. The composition may be stored at the above conditions for various periods, including 1 week, 2 weeks, 1 month, 3 months and six months.

The compositions disclosed herein can comprise the cetirizine and one or more of the listed excipients, can consist essentially of the cetirizine and one or more of the listed excipients, or can consist of the cetirizine and one or more of the listed excipients.

The invention claimed is:

1. An ophthalmic composition comprising about 0.24% cetirizine by weight and one or more pharmaceutically acceptable excipients, wherein said composition is free of benzalkonium chloride.

2. The ophthalmic composition of claim 1, wherein the cetirizine is in the form of cetirizine hydrochloride or dihydrochloride.

3. The ophthalmic composition of claim 1, wherein the one or more pharmaceutically acceptable excipients comprises viscosity-enhancer, chelating agent, tonicity agent, buffer, preservative and water.

4. The ophthalmic composition of claim 3, wherein the buffer is selected from one or more of boric acid, sodium borate, and citric acid.

5. The ophthalmic composition of claim 1, wherein the composition has a pH of about 5.5 to 7.5.

6. The ophthalmic composition of claim 1, wherein the composition is further free of dibasic sodium phosphate.

7. The ophthalmic composition of claim 3, wherein the preservative is selected from one or more of zinc chloride, sodium chlorite, sodium hydroxymethyl glycinate, polyquaternium compound such as polyquaternium-1, cationic compounds such as chlorhexidine gluconate, p-hydroxybenzoates such as methyl p-hydroxybenzoate, ethyl p-hydroxybenzoate, propyl p-hydroxybenzoate and butyl phydroxybenzoate, alcohol compounds such as phenylethyl alcohol, benzyl alcohol and chlorobutanol, sodium dehydroacetate, thiomersal and alone or in combination thereof.

8. The ophthalmic composition of claim 7, wherein the preservative is selected from one or more of zinc chloride, sodium chlorite, sodium hydroxymethyl glycinate, polyquaternium-1 and phenylethyl alcohol.

9. The ophthalmic composition of claim 1, wherein the composition comprises about 0.24% cetirizine, about 1% polyethylene glycol, about 0.25% hypromellose, about 0.1% polysorbate 80, about 1.8% glycerin, about 0.025% edetate sodium, about 0.005% zinc chloride, about 0.2% boric acid, 0.2% sodium borate and water.

10. The ophthalmic composition of claim 1, wherein the composition comprises about 0.24% cetirizine, about 1% polyethylene glycol, about 0.25% hypromellose, about 0.1% polysorbate 80, about 1.8% glycerin, about 0.025% edetate sodium, about 0.001% sodium chlorite, about 0.2% boric acid, 0.2% sodium borate and water.

11. The ophthalmic composition of claim 1, wherein the composition comprises about 0.24% cetirizine, about 1% polyethylene glycol, about 0.25% hypromellose, about 0.1% polysorbate 80, about 1.8% glycerin, about 0.025% edetate sodium, about 0.001% polyquaternium-1, about 0.2% boric acid, 0.2% sodium borate and water.

12. The ophthalmic composition of claim 1, wherein the composition comprises about 0.24% cetirizine, about 1% polyethylene glycol, about 0.25% hypromellose, about 0.1% polysorbate 80, about 1.8% glycerin, about 0.025% edetate sodium, about 0.25% phenylethyl alcohol, about 0.2% boric acid, 0.2% sodium borate and water.

13. The ophthalmic composition of claim 1, wherein the composition comprises about 0.24% cetirizine, about 1% polyethylene glycol, about 0.25% hypromellose, about 0.1% polysorbate 80, about 1.8% glycerin, about 0.025% edetate sodium, about 0.002% sodium hydroxymethyl glycinate, about 0.2% boric acid, 0.2% sodium borate and water.

14. The ophthalmic composition of claim 1, wherein the composition is a solution and includes instructions to apply once or twice daily.

15. The ophthalmic composition of claim 1, wherein the composition is filled in a preservative free bottle.

16. An ophthalmic composition comprising about 0.24% cetirizine by weight and one or more pharmaceutically acceptable excipients, wherein said composition is free of dibasic sodium phosphate.

17. The ophthalmic composition of claim 16, wherein the composition is free of benzalkonium chloride or contains less than 0.005% benzalkonium chloride.

18. The ophthalmic composition of claim 16, wherein the composition comprise non-toxic preservative selected from one or more of zinc chloride, sodium chlorite, sodium hydroxymethyl glycinate, polyquaternium-1 and phenylethyl alcohol.

19. An ophthalmic composition in the form of a solution consisting of about 0.24% cetirizine by weight as the sole active ingredient and comprising one or more pharmaceutically acceptable excipients, wherein said composition is free of dibasic sodium phosphate and benzalkonium chloride.

20. The ophthalmic composition of claim 19, wherein the one or more pharmaceutically acceptable excipients comprises about 1% polyethylene glycol, about 0.25% hypromellose, about 0.1% polysorbate 80, about 1.8% glycerin, about 0.025% edetate sodium, about 0.002% sodium hydroxymethyl glycinate, about 0.2% boric acid, 0.2% sodium borate and the remainder water.
